(19) United States
(12) Patent Application Publication
(10) Pub. No.: US 2025/0283025 A1
(43) Pub. Date: Sep. 11, 2025

(54) ASEPTIC SAMPLING DEVICE

(71) Applicant: BECARV, Tintigny (BE)

(72) Inventors: Michel Papleux, Glabais (BE); Etienne Pepinster, Gelbressee (BE)

(21) Appl. No.: 18/860,097

(22) PCT Filed: Apr. 25, 2023
(86) PCT No.: PCT/EP2023/060761

§ 371 Date: Oct. 25, 2024

(30) Foreign Application Priority Data
Apr. 28, 2022 (EP) 22170591.6

(51) Int. Cl. C12M 1/32; C12M 1/12

(52) U.S. Cl. CPC C12M 33/06; C12M 37/00

(57) ABSTRACT

The invention relates to an aseptic sampling device comprising:
A sampling means (1) being configured to aseptically collect a raw material (2) which is contained in a first container (3) of a predetermined volume, and
A receiving chamber (4) which has a confinable space (5) and being arranged to allow decontamination thereof via the passage of at least one fluid in said confinable space (5), said receiving chamber (4) being provided with a closable opening (6), a receiving means (7) which can be oriented in the confinable space (5).

The present invention relates to an aseptic sampling device configured to allow a proliferation of a raw material (in a culture medium) or to allow cell or microbial culture.

Generally, sampling devices are used in the pharmaceutical industry or in the field of biotechnologies to collect and transfer a raw material that can proliferate in a culture medium. This raw material is often in a solution, suspension, emulsion or in the form of a supernatant or plasma. It is therefore collected from a container, for example using a syringe or a pipette, and optionally transferred to another container in order to allow proliferation in a culture medium.

Therefore, the raw material can be collected and transferred from one container to another depending on the intended purpose in the technical field in question. In some fields of application such as those mentioned above, it is necessary for the raw material to be able to be aseptically collected from a container and optionally transferred aseptically to another container, in particular when a culture (cell or bacterial) is foreseen. These sampling means are a keystone of these pharmaceutical and biotechnology industries since they are part of the first step of the process. This step is crucial since it requires being able to aseptically collect and transfer a raw material, which influences the rest of the process.

More specifically, the aseptic sampling of a raw material is essential in different fields of application, in particular whenever a raw material is predisposed to being able to proliferate in a culture medium. This is commonly called “cellular reproduction/amplification” or “bacterial culture”. Among these culture techniques, the person skilled in the art is also familiar with the culture of infectious agents.

Another example is human mesenchymal stem cells (hMSCs), which are multipotent cells that can also proliferate (like undifferentiated cells). These can differentiate into different types of cells such as bone marrow, cartilage, tendon, muscle or neuronal cells. hMSCs have many therapeutic benefits such as cell therapy or tissue regeneration, and are being widely researched.

Another example where the reproduction of a raw material can be prudent is in the field of cellular immunotherapy, involving treatment which consists of producing CAR-T cells (“Chimeric Antigenic Receptor-T”) which aims to fight cancer by relying on the patient's own immune system. Once produced, the CAR-T cells are multiplied with the aim of being administered to the patient.

In addition, culturing can allow the production of vaccines or an application in the field of gene therapy.

Currently, cellular or bacterial reproduction or the culture of infectious agents is a major asset in the development of the biotechnology sector, to name but a few examples.

Typically, a culturing of cells or infectious agents can be carried out by collecting the raw material in small preservation containers. These preservation containers may be ampoules or cryotubes for cells, which are generally preserved at very low temperatures (between −70° C. and −196° C.). This process called cryopreservation allows most biological activities and chemical reactions to be suspended and therefore allows contents to be preserved. It is clear that, in the case of dilution of the raw material or formulation, a temperature between −4° C. and room temperature will be sufficient.

The cells or infectious agents are generally transferred to larger containers, where the environmental conditions conducive to their growth and reproduction are present. These culture containers can be petri dishes, flasks containing culture medium or even bioreactors containing a culture medium. This allows a large quantity of cells or infectious agents to be obtained from a small volume in order to allow the production of molecules, drugs, vaccines, genetically modified organisms or the implementation of gene therapy in order to treat certain genetic diseases.

It can be assumed that the protocol for sampling and culturing cells or infectious agents in the pharmaceutical industry or in the field of biotechnologies is relatively demanding given that the sampling step is the very first step to allow for effective culturing free of germs and unwanted bacteria in the culture medium.

However, the raw material is currently extracted manually with a syringe in a laminar flow enclosure. The user disinfects the preservation container (vial) with an alcohol wipe, for example, and then opens the glass container, for example by breaking its head, to manually collect the suspension using a syringe, which is then transferred to another container, for example for culturing.

With this type of handling, it cannot be ruled out that the user may contaminate the environment around the solution to be collected, particularly when this is in contact with this environment. The use of laminar flow cannot completely rule out the absence of specific and biological contamination due to its design.

Unfortunately, each step explained above (decontamination, opening, sampling and transfer) could lead to the introduction of contaminants. Therefore, an insufficiently decontaminated surface, human handling error or a failure in the laminar flow device leads to contamination. This results in significant delays and additional costs, with the culture then becoming unusable. In a market, often with tight flows, it is increasingly difficult to ensure that all these steps are carried out aseptically. Also, the use of laminar flow in a special enclosure is expensive for the user and does not guarantee aseptic sampling in a safe and systematic manner in the context of industrial production.

In addition, these methods can generate significant risks for the handler who may be exposed to highly contagious and pathogenic infectious agents during the various handling operations.

For these reasons, there is an identified need to be able to ensure aseptic sampling that is reliable, efficient, simple and quick, while reducing the risks of contaminants being exchanged from the user to the solution/suspension to be collected or vice versa.

There is therefore an identified need to provide a device which makes it possible to collect raw material from a first (preservation) container easily, efficiently and reliably, while limiting the risks of contamination as much as possible, as mentioned above.

To solve this problem, the present invention provides an aseptic sampling device for a proliferation of a raw material (2) (in a culture medium) or for cell or microbial culture, the device comprising:
A sampling means (1) for a raw material (2), preferably in a solution, configured to proliferate in a culture medium, said sampling means (1) being configured to aseptically collect the raw material (2) which is contained in a first container (3) of a predetermined volume, and
A receiving chamber (4) which has a confinable space (5) and which is configured to allow decontamination thereof via the passage of at least one fluid in said confinable space (5), said receiving chamber (4) being provided with:
   a closable opening (6) configured for the passage of said first container (3) in said confinable space (5) and, when it is closed, it is configured to confine the receiving chamber (4),
   a receiving means (7) orientable in the confinable space (5) and being configured to receive said first container (3) and to position it in the direction of said sampling means (1), the latter being configured to aseptically collect said solution (2) contained in said first container (3) and configured to aseptically transfer it to a second container (8) having a volume greater than the volume of said first container (3).

The device according to this invention makes it possible to aseptically collect a raw material (2), preferably in a solution, from a first container (3) (preferably for preservation) of a predetermined volume. This makes it possible to minimise the risks of contamination of both the raw material (2) (in a solution) to be collected and the user of the device as much as possible, with attractive production yields.

The device has the advantage of being able to be a single-use device, simple to use and less expensive than current solutions, in particular those requiring the use of a laminar flow enclosure. The device according to the invention is portable, which facilitates its implementation on site (industrial).

Also, the device according to the invention is configured so as to be able to be selectively confinable.

Indeed, the receiving chamber (4) can be confined by closing the closable opening (6), via a closing element, for example using a stopper, a membrane, a septum, a valve, etc. This makes it possible to choose when the device should be confined or not depending on the handling step.

When the receiving chamber (4) has a confined space (5), it is also in an aseptic environment.

According to the device of this invention, the sampling means (1) can be located either in the confinable space (5) and therefore in the receiving chamber (4), or outside the receiving chamber (4).

In the first case, the confinable space (5) extends to the sampling means (1). Therefore, the receiving chamber (4) and the sampling means (1) can then be confined by closing the closable opening (6), via a closing element, for example using a stopper, a membrane, a septum, a valve. This makes it possible to choose when the device should be confined or not depending on the handling step. The sampling means (1) and the receiving chamber (4) can therefore be confined and in an aseptic environment.

Alternatively, the device according to this invention also covers the option of having the sampling means (1) located next to (adjacent to) the confinable space (5), i.e. outside the receiving chamber (4). The sampling means (1) then forms a first confined part of the device of this invention and the receiving chamber (4) forms the second confinable part of the device according to the invention. The sampling means (1) being preferably directly (configured to be) connected (in fluid communication) to the receiving chamber (4) or indirectly connected thereto by means of an intermediate (fluid) connection element. The device according to the invention may comprise several parts.

This alternative option provided by the device according to the invention also makes it possible to selectively confine certain parts of the device. Therefore, the sampling means (1) is located in a confined and sealed space (the first part), adjacent to that of the receiving chamber (4) which is located in the second confinable part of the device. In this embodiment, it is then possible to confine (or not) the receiving chamber (4), while keeping the sampling means (1) in a confined and sealed space throughout handling and according to requirements. Preferably, fluid communication is therefore possible between the receiving chamber (4) and the sampling means (1) according to this alternative mode.

Therefore, the receiving chamber (4) can be confined by closing the closable opening (6), via a closing element, for example using a stopper, a membrane, a septum, a (solenoid) valve, etc.

These embodiments which can be combined with each other offer flexibility to the user, which is particularly advantageous.

The receiving chamber of the device according to the invention can also be decontaminated at any time in an easy and efficient manner.

Therefore, the device according to this invention makes it possible to have a closed (confined) system which makes it possible to both aseptically collect and transfer a raw material (2) which can proliferate in a culture medium.

In view of the above, the sampling device of this invention provides an effective, simple and reliable solution for aseptically collecting (and optionally transferring) a raw material (2) which is configured to proliferate in a culture medium.

In a preferred embodiment according to this invention, said sampling means (1) is adjacent to said confinable space (5) of said receiving chamber (4) before sampling, and is preferably in a confined and sealed medium. This embodiment makes it possible to facilitate the sampling of the raw material (2) and effectively ensure aseptic sampling. The sampling means is preferably located outside the receiving chamber (4) before sampling. The sampling means (1) then advantageously forms a first confined part of the device of this invention and the receiving chamber (4) forms the second confinable part of the device according to the invention. The sampling means (1) being preferably directly (configured to be) connected to the receiving chamber (4) or (configured to) be indirectly connected to the latter by means of an intermediate (fluid) connection element. The device according to the invention may comprise several parts.

This alternative option provided by the device according to the invention makes it possible to confine certain parts of the device more selectively. Therefore, the sampling means (1) is located in a confined and sealed space (the first part) relative to the receiving chamber (4) and adjacent to it (second part of the device). In this embodiment, it is then possible to confine (or not) the receiving chamber (4), while keeping the sampling means (1) in a confined and sealed space throughout handling and according to requirements. Preferably, a fluid communication is established between the receiving chamber (4) and the sampling means (1) according to this advantageous embodiment.

Therefore, the sampling means (1) can be confined independently of the confinable space (5) of the receiving chamber (4). This makes it possible to reduce the possible risks of contamination and to allow careful control of the sampling means (1) and the confinable space (5) of the receiving chamber (4).

Also, the sampling means (1) is in an aseptic environment.

Therefore, the receiving chamber (4) can be confined by closing the closable opening (6), via a closing element, for example using a stopper, a membrane, a septum, a (solenoid) valve, etc.

This flexibility offered to the user is particularly advantageous.

In a preferred embodiment according to this invention, said sampling means (1) is separated from the receiving chamber (4), preferably via a closable element (9) chosen from the group comprising a septum, a membrane and any other equivalent element which allows opening and closing in a sealed manner, while ensuring aseptic sampling. A solenoid valve could also be used in these preferred conditions.

A closable element (9) makes it possible to isolate the receiving chamber (4) from the sampling device (1) and at the same time facilitates access to the confinable space (5). This makes it possible to further reduce the risks of contamination. It is also possible to carry out several rinses and/or decontaminations of the space (5) when it is confined. A fluid communication between the sampling means (1) and the receiving chamber (4) is preferably established in order to facilitate the sampling of the raw material (2).

The preceding paragraphs and the related description relating to the embodiment wherein the sampling means (1) is adjacent to the receiving chamber (4) are also applicable here.

Preferably, during said sampling, a part of said sampling means (1) is located in the confinable space (5) of the receiving chamber (4). This makes it possible to collect the raw material (2) from the first container (3), while limiting the time spent and the degree of exposure of the sampling means (1) in the confinable space (5). More precisely, this embodiment is carried out when the space (5) of the receiving chamber (4) is confined and sealed relative to the external surrounding environment.

It is preferable to avoid any introduction of the sampling means (1) into the confinable space (5) of the receiving chamber when this is open to the surrounding environment.

More preferably, the sampling means (1) is located in a sampling chamber (11) which has a guide means (12) for said sampling means (1). This makes it possible to guide the sampling device (1) more easily in order to facilitate handling during sampling.

Advantageously, said guide means (12) is configured to move said sampling means (1), preferably, along a median axis of said sampling chamber (11) towards said receiving chamber (4). This makes it even easier to guide the sampling device (1) in a predetermined sampling direction.

According to a particularly preferred embodiment, the sampling chamber (11) has a flexible body (11a) which has a resting position and a sampling position. Therefore, when the flexible body (11a) is in the resting position, the entire sampling means (1) is located in the sampling chamber (11). When the flexible body (11a) is in the sampling position, the sampling means (1) is moved into the confined space (5) of the receiving chamber (4) to be in contact with the raw material (2) to be collected (in a solution). When sampling is carried out, the flexible body (11a) is arranged to move back to the resting position.

By means of a pumping or suction or piston system or any other sampling system which ensures aseptic sampling, the raw material (2) can preferably be collected and transferred to the second container (8). The latter may comprise a culture medium which will be mixed with the raw material (2) so as to allow an amplification of the raw material (2).

Alternatively, this flexible body (11a) may also be a piston which also makes it possible to provide a confined environment in which the sampling means is located. The operation indicated above is then similar with regard to the rest and sampling positions.

Advantageously, the guide means (12) has a groove (12a) which extends longitudinally on an outer surface (12b) of said guide means (12a) to make said sampling means (1) slide so that one of the ends (1a) of said sampling means (1) is introduced into said receiving chamber (4).

Even more advantageously, said sampling means (1) has a body (13), preferably a pump body (13), configured to allow the passage of said raw material (2) and which is provided at one of its ends with a needle (14) or a line. The presence of a needle (14) or a channel allows the sampling means (1) to be easily inserted inside the first container (3), and is therefore a means which allows a fluid to pass, for example by suction. A needle will be preferred for efficiency because it ensures simple aseptic sampling.

Preferably, therefore, the needle (14) can be replaced by a line that would allow efficient sampling and transfer. Indeed, the needle or line plays a rather passive role in the device since it does not ensure the removal of the raw material (2) but rather provides easy and efficient access to the bottom of the first container (3) (vial). Any other equivalent means would also be suitable.

Note that the needle will be preferred for efficiency.

This type of handling can therefore be used to allow aseptic sampling in a closed/confined system (provided by the device according to the invention).

Alternatively, the sampling means (1) is located in a confined and sealed environment and the receiving chamber (4) in a separate confinable space (5).

In a preferred embodiment according to this invention, said sampling chamber (11) is confined and sealed. This embodiment makes it possible to provide the sampling means (1) with an environment isolated from that of the confined space (5) of the receiving chamber (4). Therefore, when the receiving chamber (4) is open, the sampling chamber (11) (and the sampling means (1)) remain confined and sealed, which provides freedom of action when handling the device, while avoiding any contamination of the sampling means (1).

In addition and preferably, the receiving chamber (4) has at least a part of its surface made of flexible material (15), said part being optionally adjacent to said receiving means (7), which is configured to pass through said at least one part (15) of the surface of the receiving chamber (4) made of flexible material. The flexible part (15) makes it possible to ensure that the receiving means (7) can be oriented following facilitated handling and, preferably throughout the confineable space (5). This part made of flexible material (15) advantageously allows a rotational movement, pivoting and/or a longitudinal movement of the receiving means (7). This means that it is not ruled out that the receiving chamber (4) has a part of its surface made of rigid material that is slightly flexible relative to the part made of flexible material. It is preferable to have a receiving chamber (4) that has a part of its surface that is deformable in space (part made of flexible material) and another part that is less so (part made of rigid material) in order to be able to offer some support for a part of the elements of the receiving chamber (4).

According to an advantageous embodiment, the device according to the invention comprises a second container (8) adjacent to said receiving chamber (4) and which is connected by said sampling means (1) to said receiving chamber (4). Therefore, the second container (8) is in fluid communication with the sampling means (1), which makes it possible to transfer the raw material (2) to this second container (8) which contains a culture medium suitable for cellular reproduction or any other type of amplification of a raw material (2).

Advantageously, a fluid communication can be established between the second container (8) and the sampling means (1) and the receiving chamber (4) selectively in order to minimise the presence of contaminants during handling.

Preferably, said second container (8) has a volume greater than that of the first container (3), said second container (8) being arranged to allow culturing (amplification of a raw material (2)), when said collected solution (2) from said first container (3) is transferred into said second container (8). This makes it possible, from a small container (3), to seed a larger container (8), to allow amplification of the raw material (2), in particular cellular reproduction.

In a preferred embodiment according to this invention, the receiving means (7) has a first part (7a) which extends into said confinable space (5) and a second part (7b) which extends outside said receiving chamber (4) in the form of a guide means (10), preferably along a median axis which passes through said receiving chamber (4). This allows the user to easily guide the receiving means manually or automatically by orienting this second part which is in the form of a guide means (10) outside the receiving chamber (4), without the risk of contamination.

Even more preferably, the part in the form of a guide means (10) is in fluid communication with said receiving chamber (4). This makes it possible to connect a fluid removal means (18), for example by connecting a line to one of the ends of this second part in the form of a guide means (10). Access to the receiving chamber (4) is therefore facilitated. This access is located along a median axis of the receiving chamber (4), preferably opposite the closable opening (6).

The receiving chamber (4) is advantageously arranged to be (or is) in fluid communication with at least one of the following elements or taken in combination:
A means for supplying at least one decontamination fluid (16)
A means for supplying at least one rinsing fluid (17),
A means for supplying at least one drying fluid (19),
A first means for removing at least one fluid (18), said removal means (18) being optionally directly connected to one of the ends (7c) of said receiving means (7),
And a second means for removing (drying) fluid (20)

In this way, the receiving chamber (4) can be prepared before receiving the first container (3) in order to ensure that the confinable space (5) can allow aseptic sampling to be ensured when the opening (6) is closed after introduction of the first container (3) into the confinable space.

This preferred embodiment implies that the receiving chamber (4) can be in fluid communication with at least one of the aforementioned elements (16, 17, 18, 19, 20).

Preferably, the receiving chamber (4) comprises a means (22) for opening the first container (3), the opening means is preferably located in the confinable space (5). The opening means (22) is configured to allow access to the raw material (2) of the first container (3). The opening means (22) can for example break one of the ends of the first container (3) (container which can be made of glass) or unlock/unscrew a stopper with ease, or even uncap the first container (3). This additional option linked to the presence of said opening means (22) therefore allows access to the raw material (2) contained in any type of container (3), in particular when the first container (3) is housed in the receiving chamber (4).

In a particularly preferred manner, said receiving means (7) is moulded in a single piece. Also, the receiving means can be machined from a block of plastic or metal material.

The device according to the invention can for example be manufactured by a 3D printing process or via moulding involving the use of a series of moulds.

In a preferred embodiment according to this invention, the device is a single-use device, preferably portable.

In a preferred embodiment according to this invention, the first container (3) has a volume of less than 20 ml, preferably less than 15 ml or greater than 2 ml.

The first container (3) is preferably chosen from the group consisting of an ampoule, a cryotube, a vial (which may come from the company Aseptic Technologies). The material of the first container may be made of glass or plastic.

Even more advantageously, the device according to the invention may operate using an automated or even robotic system.

Other embodiments of the method according to the invention are indicated in the appended claims.

### Definitions

The term “aseptic” refers to the conditions under which sampling and transfer actions are performed. In the context of this invention, a sampling device is provided that allows a raw material to be collected aseptically. In other words, each action that can be performed using the device according to the invention is done aseptically, which allows the environment (for example the confined space) to be kept aseptic. This also applies to the sampling means which is configured to aseptically collect and transfer the raw material.

In the context of this invention, the expression “raw material” includes the term strain or any other biological material that can proliferate in a culture medium. The “raw material” is preferably a solution, a suspension, an emulsion or in the form of a supernatant. The raw material is preferably selected from the group comprising (consisting of) cells (of human or animal origin), bacteria, infectious agents, viruses, (living) (micro)organisms, yeasts, algae, prions, and mixtures thereof. Preferably, an ingredient or excipient that can be used in the composition of sterile solutions can also constitute the raw material. Preferably, the raw material can be in solid form and then dissolved before introduction into the device according to the invention or this dissolution is carried out when the container is introduced into the device according to the invention. All the features of this paragraph can be combined with each other.

“(Micro)organism” refers to all organisms invisible to the naked eye. This term includes bacteria, protozoa, microscopic algae, microscopic fungi. This definition also includes plant, animal or human eukaryotic cells, derived from tissues, cancerous or non-cancerous cell lines. This definition must also include all infectious agents, living or inert, such as viruses or prions. The aforementioned categories may be genetically modified.

“Sealed” refers to the ability to not allow fluids (liquids, gases), dust or humidity to pass through.

The expression “confinable space” should be understood to mean that the space can be confined, in relation to the external environment, in particular external to that of the receiving chamber or with respect to other surrounding compartments. This feature may be linked to the closable opening. Therefore, when this opening is closed, the confinable space becomes confined and, preferably, also sealed. It is therefore possible to access the confinable space by means of the closable opening which leaves some flexibility to the user at the time of (manual or automatic) handling.

This confinable space is arranged to be decontaminated, in particular before sampling or before the introduction of the first container into the receiving chamber.

The expression “first container” can be interpreted in the context of this invention as “the (preservation) container” which contains the raw material to be collected. Therefore, this type of first container may be present in large quantity (in series). A plurality of first containers can therefore be addressed by this invention in view of the embodiments disclosed. Therefore, several containers can be present in the receiving chamber in order to operate the device in series or several first containers can be added individually, one after the other, in series.

The expression “second container” relates to a type of amplification container. In other words, this type of container may also be present in large quantities (in series). This allows amplification of the raw material, in particular cellular reproduction. For example, a bioreactor can act as a second container and comprise a culture medium.

As explained above, the pharmaceutical or biotechnology industry will use the sampling device according to the invention to collect and, optionally, transfer a raw material (2). The latter can proliferate in a culture medium. This raw material (2) can be in a solution, or in a suspension or in the form of a supernatant. It is therefore collected from the first container (3) with the sampling means (1), and optionally transferred to a second container (8) in order to allow proliferation in a culture medium. This is commonly called “cellular reproduction” or “bacterial culture”. Among these cultures, the person skilled in the art is also aware of the culture of infectious agents, human mesenchymal stem cells (hMSCs), CAR-T cells (i“Chimeric Antigenic Receptor-T”) which are multiplied for the purpose of being administered to the patient. In addition, culturing can be used in the production of vaccines, in the field of gene therapy.

According to this invention, it is preferably possible to use a decontamination fluid selected from the group comprising water, hydrogen peroxide, peracetic acid, and mixtures thereof.

Advantageously, the drying fluid can be compressed air.

Preferably, the rinsing fluid can be water.

Therefore, the device according to the invention is suitable for use in the context of cellular or bacterial reproduction or the culture of infectious agents, to name just a few examples.

According to a preferred embodiment of the present invention, a first container (3) comprising at least one cell in a solution (2) is introduced into the closable opening (6). The container is closed. Then, the receiving means (7) which is positioned along a median axis of the receiving chamber (4) is oriented towards the opening (6) and receives the first container (3) which is held stably in the receiving means (7). The opening (6) is closed using a closing element, such as a stopper. Therefore, the receiving chamber (4) is sealed, when the opening (6) is hermetically closed, which forms a confinable space (5) in the receiving chamber (4). The receiving chamber (4) is then decontaminated using a fluid. When the receiving chamber (4) is decontaminated, the first container (3) is oriented with the receiving means (7) towards the sampling means (1). The sampling means (1) is positioned inside the confinable space (5) until it reaches the bottom of the first container (3). It should be noted that the sampling means (1) is located in a confined environment before sampling. By means of a pumping system, the solution (2) is collected and transferred to a second container (8) which contains a culture medium.

Advantageously, the collected material in a solution is transferred aseptically to a second container (8) which has a volume greater than that of the first container (3). These steps can be carried out several times, i.e. by introducing several containers of the same type as the first container (3) in series into the receiving chamber (4).

It should be noted that the first container (3) is intended to comprise a raw material (2) configured to proliferate in a culture medium. The second container (8) is itself intended to contain culture medium and the raw material (2) contained in the first container (3).

The volume of the second container (8) in the case of cellular reproduction will preferably increase according to a complete amplification process (commonly called the “end to end” process) so as to increase the quantity of amplified material up to the desired final volume. The aim is to advantageously and ultimately provide an injectable solution, preferably for the pharmaceutical field or for biotechnological applications.

According to a preferred embodiment, the device according to the invention is therefore arranged to allow the aseptic production of an injectable solution for the pharmaceutical field or for biotechnological applications.

Alternatively, it is also possible to have a receiving chamber (4) which has several receiving means (7) so as to be able to collect raw material (2) from several containers (3).

The sampling device of this invention allows the aseptic sampling and optionally the transfer of a raw material (2) which is arranged to proliferate in a culture medium. Aseptic sampling makes it possible to avoid any contamination of the contents of the first container by unwanted contaminants, such as germs of the microorganism type. This has the advantage of increasing production yields, thanks to the limitation of raw material (2) waste resulting from potential contamination.

The device according to the present invention comprises a receiving chamber (4) that is sealed when the closable opening (6) is closed. Sealing prevents fluids (liquids or gases), dust, and moisture from passing through. This therefore aims to create a confined space (5) relative to the surrounding environment, external to the receiving chamber (4), and optionally external to the sampling means (1).

It should be noted that the user can be replaced by an automatic, robotic system, which makes it possible to dispense with the presence of the user.

Also, said receiving means (7) is orientable in the confinable space (5), which allows it to be oriented in the confinable space (5) as desired. This therefore advantageously makes it possible to position the receiving means (7) facing the closable opening (6), to orient this first container (3) towards the sampling device (1), or even preferably to make it possible to orient this first container (3) in a place in the receiving chamber (4) to optionally break one of the ends of a container (made of glass) in order to have access to its contents.

Advantageously, the device according to the invention is sterilised by gamma irradiation, preferably before any handling of the sample, or even before any introduction of the first container (3) into the device.

The device according to the invention allows an amplification or proliferation of the raw material. Preferably, it is also possible to use the device according to the invention to dilute the raw material or to do formulation or sterility tests.

Other characteristics, details and advantages of the invention will be discussed in the description given below, in a non-limiting manner and with reference to the drawings and examples.

The references of each element included in this invention are as follows:
Sampling means (1)
End of the sampling means (1a)
Raw material to be collected (2)
First container (3)
Proximal end of the first container (3a)
Receiving chamber (4)
Confinable space (5)
Closable opening (6)
Receiving means (7)
First part of the receiving means (7a)
Second part of the receiving means (7b)
Distal end of the receiving means (7c)
Second container (8)
Resealable element (9)—providing sealing
Guide means (10)—second part of the receiving means (7b)
Sampling chamber (11)
Flexible part (11a) of the sampling chamber (11)
Guide means (12) of the sampling means (1)
Groove (12a) of the guide means (12)
Outer surface (12b) of the guide means (12)
Body (13) of the sampling means (1)
Needle (14) of the sampling means (1)
Flexible part (15) of the receiving chamber (4)
Means for supplying at least one decontamination fluid (16)
Fluid connection of at least one decontamination fluid (16a)
Means for supplying at least one rinsing fluid (17, 27)
Fluid connection of at least one rinsing fluid (17a)
First fluid removal means (18)
Means for supplying a drying fluid provided with a filter membrane and a
hydrophobic filter (19)
Fluid connection of at least one drying fluid (19a)
Fluid connection for decontamination of the fluid connection of at least
one drying fluid (19b)
Second fluid removal means (20)
Rigid part 21 (rigidity defined relative to the flexible part (15)) of the
receiving chamber (4)
Opening means (22) of the first container (3)
Means for receiving at least one filter purge fluid (24)
Fluid connection of at least one filter purge fluid (24a)
Means for storing used fluids (25)
Filtration means (26) with a hydrophilic filter
Means for supplying at least one rinsing fluid (27) for the sampling means (1)
Fluid connection of at least one rinsing fluid (27a) for the sampling means (1)
Sampling line (27b, 27c)
Means for venting the container (8) and for suctioning the raw material (2)
Pumping means, pumps (A)
Hydrophobic or hydrophilic filter (F) depending on the intended use

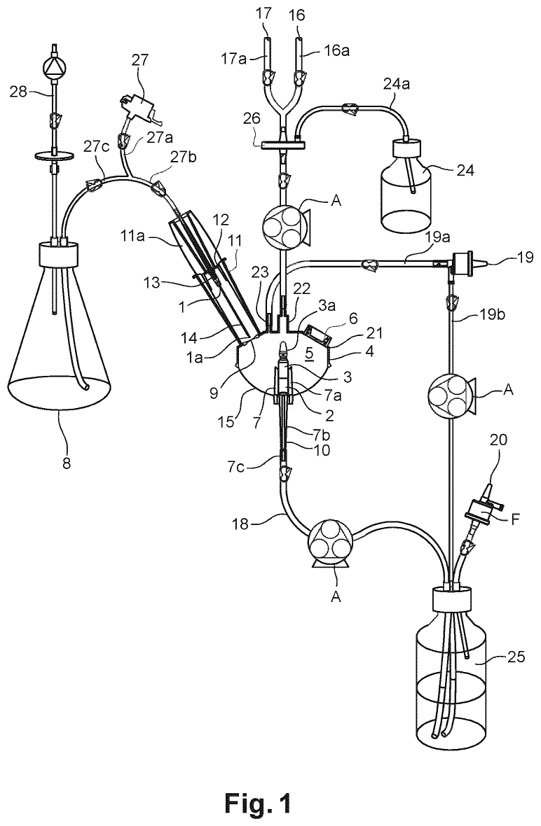

FIG. 1 is a schematic view of an embodiment of the device according to this invention.

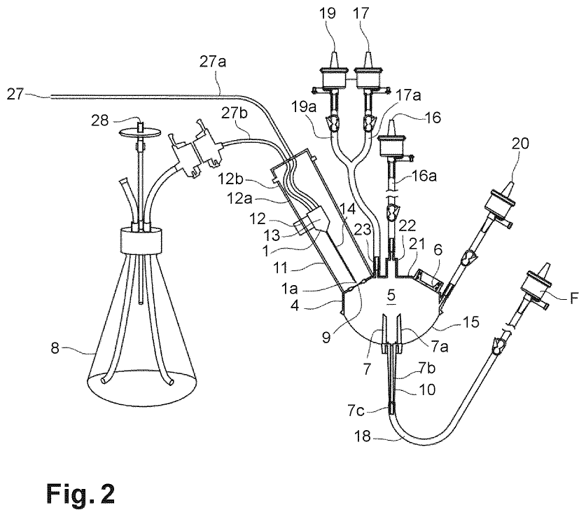

FIG. 2 is a schematic view of a preferred embodiment of the device according to the present invention.

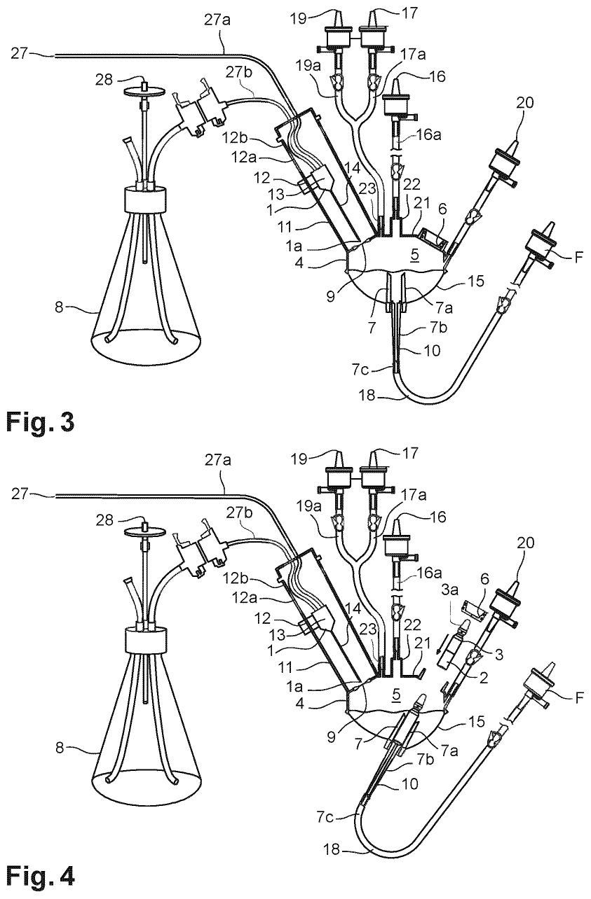

FIGS. 3 to 10 are illustrations of the device of FIG. 2 according to different handling steps.

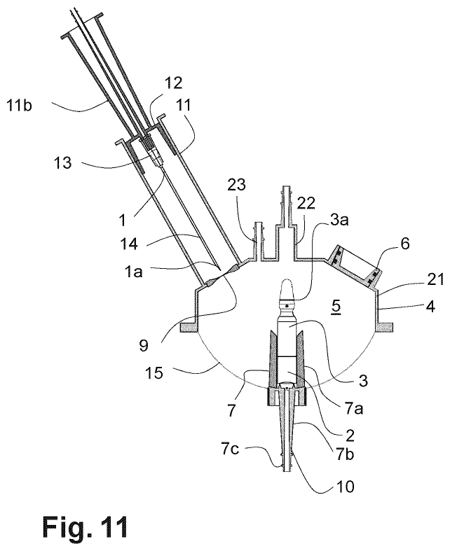

FIG. 11 is a schematic view of an embodiment of the device according to this invention.

In the figures, identical or similar elements have the same references.

Other characteristics and advantages of this invention will be set forth in the non-limiting description below, and with reference to the drawings and the examples.

FIG. 1 illustrates an aseptic sampling device according to an embodiment of the invention which is arranged to allow the sampling of a raw material (2). This raw material (2) may be a suspended cell or a (micro)organism.

The device of FIG. 1 comprises a means for sampling (1) a raw material in a solution and a sealed receiving chamber (4) which has a confined space (5). The sampling means (1) is adjacent to the receiving chamber (4) which is provided with an opening (6) closed by a stopper and a receiving means (7). In addition, it appears that the sampling means (1) is separated from the receiving chamber (4) via a closable element, in this case via a septum (9). Also, the sampling means (1) is located in a sampling chamber (11) which has a guide means (12). This guide means (12) is in the form of a cone which is connected to the sampling means (1) at the body (13), for example a tip (14), which is provided at one of its ends with a needle (14) and, optionally at the other end with a pump body (or a line).

The sampling means (1) therefore has a sealed body (13) provided at one of its ends with a needle (or any other means capable of allowing the raw material present in the first container (3) to pass through). The body (13) is also provided at the other end, opposite the needle, with a line (27b). Therefore, the body (13), the needle (14) and the line (27b) are in fluid communication.

The sampling chamber (11) is confined and sealed, which is therefore also the case for the sampling means (1) which is located in a confined environment and separated from the confined space (5) of the receiving chamber (4).

Also, the sampling chamber (11) comprises a flexible body (11a), which preferably functions like an accordion, which has a rest position (expanded accordion shape) and a sampling position (compressed accordion shape). Therefore, when the flexible body (11a) is in the rest position, the entire sampling means (1) is located in the sampling chamber (11) as illustrated in FIG. 1. When the flexible body (11a) is in the sampling position, it is in a position where it is folded back on itself (like an accordion in a compressed position) and the sampling means (1) is then located in the confined space (5) of the receiving chamber (4). When the sample is taken, the flexible body (11a) is arranged to return to the rest position (with the accordion in the expanded position).

Alternatively, this flexible body (11a) may also be a piston which also makes it possible to provide a confined environment in which the sampling means is located. The operation indicated above is then similar with regard to the rest and sampling positions. Such an embodiment is illustrated in FIG. 11.

The receiving chamber (4) has at least part of its surface made of flexible material (15), said part being optionally adjacent to said receiving means (7), which passes through this part (15) in a sealed manner, preferably along a median axis of the receiving chamber (4). FIG. 1 shows a receiving chamber (4) which has part of its surface made of flexible material (15) and another part made of rigid material (21) (this rigidity being defined relative to the flexible material). Therefore, the surface made of flexible material (15) can be made of a malleable plastic and the more rigid material (15) can be made of elastomer, preferably rubber (less malleable).

The receiving means (7) of the receiving chamber (4) comprises a first part (7a) located in the confinable space (5) which stably holds (without manual intervention) the first container (3) which contains a raw material (2) to be collected, for example suspended cells (2). The first container (3) has a proximal part 3a (made of glass) and is closed when it is in the confined space (5). This first part (7a) is connected to a second part (7b) which is located outside the receiving chamber (4). This second part (7b) extends outside said receiving chamber (4) in the form of a guide means (10), which can be oriented relative to a median axis which passes through said receiving chamber (4).

In addition, the second part (7b) of the receiving means (7) has a distal end (7c) connected to a first means for removing at least one fluid (18). Therefore, the receiving means (7) is in fluid communication with the confined space (5) of the receiving chamber (4).

Alternatively, the distal end (7c) is closed, without being connected to the first means for removing at least one fluid (18). In this embodiment, the means for removing at least one fluid (18) is located elsewhere on the receiving chamber (4), so as to be separated from the distal end (7c) of the receiving means (7).

Also, the receiving means (7) is located on the part of the receiving chamber (4) which is made of flexible material (15).

The receiving chamber (4) further comprises, and preferably on its rigid material part, the closed opening (6), a means (22) for opening the first container (3) and a fluid connection (23) of a fluid.

The opening means (22) is capable of receiving the proximal part (3a) of the first container (3) and is arranged to break the proximal part (3a) of the first container (3) via a pivoting movement.

Depending on the type of means for closing the first container (3), the opening means (22) will have a shape and a function that will allow aseptic access to the first container (3). This type of closing means may be a stopper, a membrane, a capsule, etc.

It is clear that when the first container (3) is provided with a septum at the proximal end (3a) of the first container (3), the opening means (22) is not used (or even present) in the receiving chamber (4).

As illustrated in FIG. 1, the opening means (22) can also be connected to a fluid connection which is connected to a filtration means (26). This filtration means (26) makes it possible to filter the fluids, here the rinsing and/or decontamination fluids to limit contamination and dirtying of the receiving chamber (4). This filtration means (26) is connected to a fluid connection of at least one filter purge fluid (24a) which is in the form of a line (24a) which lands in a container (24) acting as a means for receiving at least one filter purge fluid (24).

The filtration means (26) is also connected to a means for supplying a decontamination fluid (hydrogen peroxide) (16) via a decontamination fluid connection (16a). In addition, the filtration means (26) is connected to a means for supplying a rinsing fluid (water) (17) via a rinsing fluid connection (17a).

Alternatively, when the opening means (22) is not present, the supply connections (16, 17), the filtration means (26) and their respective lines (16a, 17a, 24a) are configured to be in fluid communication with the receiving chamber (4).

In the embodiment illustrated in FIG. 1, the receiving chamber (4) is also fluidly connected to a means for supplying a drying fluid (air) (19). Therefore the tip (23) of the receiving chamber (4) is connected by a line (19a) to the drying fluid (air) supply means (19). Then, the drying fluid supply means (19) is also connected to a line (19b) which allows, via a pump (A), the fluids to be removed from the receiving chamber (4) following drying to a receiving container (by disposal, container (25)).

The illustrated device also comprises a fluid (here air) removal means (20) which can for example be located in the container (25) or elsewhere depending on the arrangement of all the elements forming the device according to the invention.

In addition, the body (13), the needle (14) and the line (27b) are in fluid communication. The sampling line (27b, 27c) makes it possible to transfer the raw material (2) in a solution (suspended cell) to a second container (8). It is also possible to rinse the sampling means (1) using a means for supplying a rinsing fluid (27). The device may also include a removal means (28).

In practice, the second container may contain a culture medium (not illustrated).

The second container (8), adjacent to said receiving chamber (4), is connected by said sampling means (1) to said receiving chamber (4), via which the raw material (2) of interest is transferred.

The second container (8) has a volume greater than that of the first container (3) and is configured to allow culturing, when said raw material (2) collected from said first container (3) is transferred into said second container (8).

FIG. 1 therefore comprises several lines provided to allow at least one fluid to pass (16a, 17a, 18, 19a, 19b, 24a, 27a, 27b, 27c, 28), several pumps (A), as well as elements (19, 20, 24, 25, 26, 27) commonly used in the technical field of this invention and which can be connected to the device of the invention in several ways.

During operation, a decontamination liquid (hydrogen peroxide) is supplied by the fluid supply means (16), until the receiving chamber (4) is partially filled.

Then, the stopper of the opening (6) is removed and an aseptic vial (3) (provided by the company Aseptic Technologies) containing suspended cells (2) is introduced into the receiving chamber (4), in which the receiving means (7) is oriented at its second part (7b) towards the opening (6). Therefore, the vial (3) is stably housed in the receiving means (7). When the receiving means (7) is oriented, the flexible surface (15) of the receiving chamber (4) deforms to allow adequate orientation. When the vial (3) is held in the receiving means (7), the latter is positioned vertically along a median axis of the receiving chamber (4).

A decontamination step is then carried out by supplying the receiving chamber (4) with decontamination fluid (hydrogen peroxide) until it is completely filled with decontamination fluid. As soon as decontamination is complete, emptying is carried out in order to eliminate the decontamination fluid (line (18) in the disposal container (25).

After the decontamination step, a complete filling of the receiving chamber (4) is carried out with a rinsing fluid (water) using the rinsing fluid supply means (17) in order to eliminate all traces of decontamination fluid. An emptying step towards the disposal container (25) then follows. Preferably, this rinsing step is dynamic in that it eliminates the decontamination liquid and is therefore not limited to simply diluting it.

A drying step then takes place via the drying fluid supply means (19), preferably by introducing compressed air.

When the vial (3) has to be broken at its proximal end (3a), it is oriented in the opening means (22) which will subsequently allow a rotation/pivoting movement to break this end in order to allow access to the suspended cells (2) to be collected. This step is optional and not necessary when access to the raw material (2) to be collected does not require breaking a part of the container (3). For example, when the container is provided with a septum or any other resealable element that allows access to the contents of the vial without breakage.

Then, the receiving means (7) housing the open vial is oriented towards the sampling means (1), preferably along a median axis of the sampling chamber. Therefore, the opening of the vial is located opposite the septum (9).

As explained above, the flexible body (11a) of the sampling chamber will allow the needle (14) of the sampling means (1) to be inserted into the confined space (5) of the receiving chamber (4) by moving from a rest position (expanded accordion shape, as illustrated in FIG. 1) to a sampling position (compressed accordion shape). When the flexible body (11a) is in the sampling position, it is in a position where it is folded back on itself (like an accordion in a compressed position) and the sampling means (1) is then located in the confined space (5) of the receiving chamber (4), more precisely at the bottom of the vial (3) ready to collect the suspended cells.

By means of a pumping or suction or piston system or any other sampling system that guarantees aseptic sampling, the suspended cells are therefore collected and transferred to the second container (8). The latter comprises a culture medium that will be mixed with the suspended cells so as to allow cell reproduction. When the sample is taken, the flexible body (11a) is arranged to return to the rest position (with the accordion in the expanded position).

Therefore, the needle (14) can be replaced by a conduit that would allow efficient sampling and transfer. Indeed, the needle has a rather passive role in the device since it does not ensure the removal of the suspended cells but rather allows easy and efficient access to the bottom of the vial. Any other equivalent means would also be suitable.

This type of handling can therefore be used to generally allow an aseptic sampling means in a closed system (provided by the device according to the invention).

“End-to-end” type processes can also integrate the device according to the invention and consist of integrating all the steps of a production process from the raw material to the finished product. Therefore, the device according to the invention makes it possible to integrate the first step of a process known as “end-to-end”.

FIG. 2 is similar to FIG. 1 and illustrates another embodiment of the device according to the invention by using part of the references of FIG. 1, except that the sampling means (1) is different. The sampling means 1 is located in a sampling chamber (11) which has a guide means (12) which makes it possible to move said sampling means (1), preferably along a median axis of said sampling chamber (11) towards said receiving chamber (4). The guide means (12) has a groove (12a) which extends longitudinally on an outer surface (12b) of said guide means (12) to make said sampling means (1) slide so that one of the ends (1a) of said sampling means (1) is introduced into said receiving chamber (4). In this case, the needle can therefore be introduced into the confined space (5) of the receiving chamber (4).

As illustrated, the body (13) is provided with a guide means (12), in the form of a protrusion, which extends outside the sampling chamber (11).

In this embodiment, a flexible body (11a) is not necessary, which makes it possible to have a sampling chamber (11) that is partly rigid.

The operation of the device illustrated in FIG. 1 described above is also applicable here.

It should be noted that concerning the different handling steps related to decontamination, rinsing and drying. The person skilled in the art will be able to combine these steps as desired depending on requirements during handling.

FIGS. 3-10 illustrate how the device can be used, in particular when rinsing and/or decontamination and/or drying takes place.

These FIGS. 2-10 can also be understood as sequentially illustrating how the device is implemented.

FIG. 3 illustrates the device according to a preferred embodiment when a decontamination fluid (such as hydrogen peroxide) is introduced into the receiving chamber (4) which is partially filled with decontamination liquid. This fluid may also be a rinsing fluid, such as water.

FIG. 4 illustrates the device according to an advantageous embodiment, wherein a step of positioning the first container (3) in the receiving chamber (4) is carried out by said receiving means (7) which is oriented towards the closable opening (6).

According to a particularly advantageous embodiment (not illustrated), wherein the first container (3) is positioned along a median axis of the receiving chamber (4), in a vertical position, when a decontamination fluid (such as hydrogen peroxide) partially or completely fills the receiving chamber (4). This fluid may be a rinsing fluid (such as water). This step may be located after that illustrated in FIG. 4.

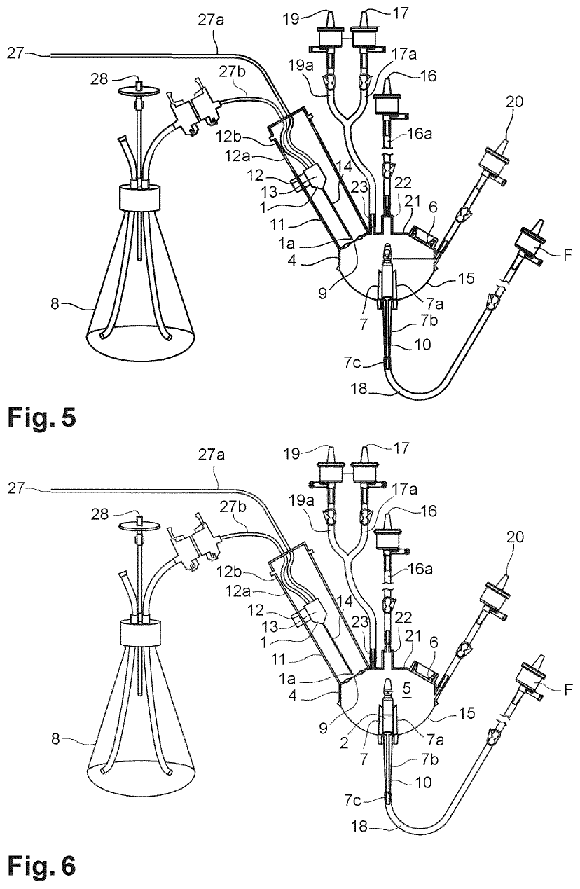

FIG. 5 illustrates a complete decontamination step by filling the entire receiving chamber (4) with a decontamination fluid.

FIG. 6 illustrates an advantageous embodiment, wherein a step of emptying the decontamination (or rinsing) fluid from the receiving chamber (4) is carried out.

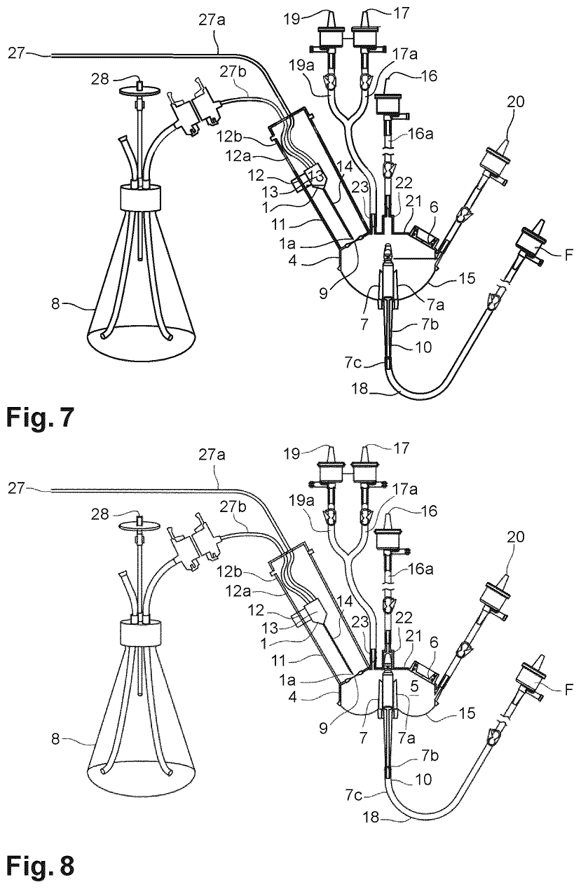

FIG. 7 illustrates a complete rinsing step by filling the entire receiving chamber (4) with a rinsing fluid.

FIG. 8 illustrates an embodiment wherein the flexibility of a part of the receiving chamber (4) is illustrated. A step of opening the first container (3) is carried out after positioning the first container (3) in the opening means (22).

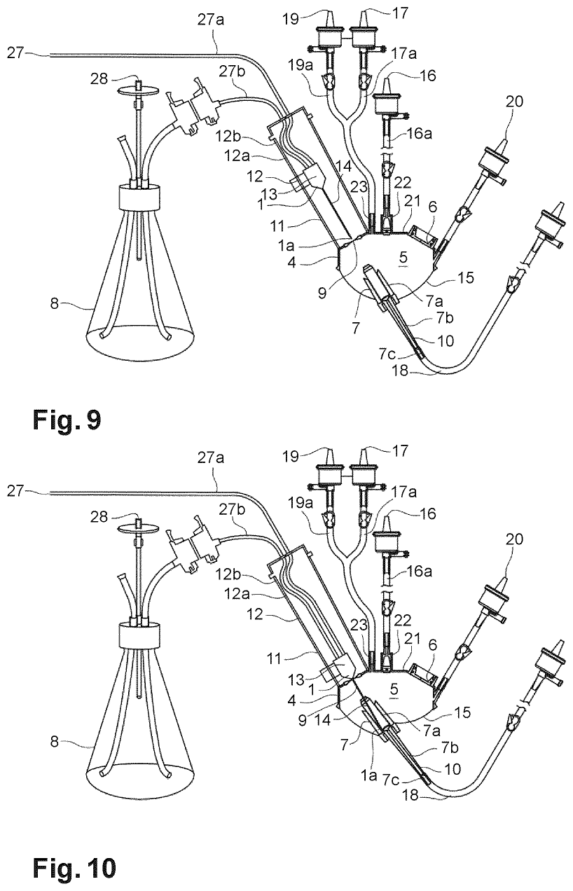

FIG. 9 illustrates a particular embodiment, wherein a step of positioning the opening of the first container (3) opposite the sampling means (1) is carried out by pivoting the receiving means (7).

FIG. 10 illustrates a preferred embodiment wherein the sampling means (1) is placed inside the first container (3) to allow sampling.

FIG. 11 illustrates an additional embodiment of the device according to the invention which includes the elements described in FIG. 1, except for the sampling chamber which comprises a rigid body (11b) which has a rest position and a sampling position. Therefore, when the rigid body (11b) is in the rest position, the entire sampling means (1) is located in the sampling chamber (11). When the rigid body (11b) is in the sampling position, the sampling means (1) is moved into the confined space (5) of the receiving chamber (4) to be in contact with the raw material (2) to be collected (in a solution). When sampling is carried out, the rigid body (11b) is arranged to move back to the rest position.

In a preferred embodiment, the rigid body (11b) is a piston, as illustrated in FIG. 11, which also provides a confined space to receive the sampling device.

In another preferred embodiment, the sampling chamber (11) is provided with a stopper on one of its internal walls, making it possible to prevent the rigid body (11b) from accidentally exiting the sampling chamber (11) when it is in the rest position.

In a more preferred embodiment, the guide means (12) comes into contact with the stopper in order to prevent the rigid body (11b) from accidentally coming out of the sampling chamber (11) when it is in the rest position.

The embodiment illustrated in FIG. 8 is not essential when the first container (3) is provided with a resealable element, such as a septum or any equivalent element.

In the figures, identical or similar elements have the same references.

In the context of this invention, any singular article such as for example, “a”, “an”, “the”, “of”, “of” can be replaced by an article which designates a plural such as for example “at least 2”, “at least 3”, “several” etc.

The word “comprise”, “contains” or any equivalent term or derivatives can be replaced by “consisting of” in order to define a list or exclusive selection possibilities so as not to include other elements not mentioned in the expression used.

It is understood that this invention is in no way limited to the embodiments described above and that many modifications can be made thereto without departing from the scope of the appended claims.

1. An aseptic sampling device for the proliferation of a raw material in a culture medium or for cellular or microbial culture, the aseptic sampling device comprising:
A sampling means for a raw material, preferably in a solution, arranged to proliferate in a culture medium, said sampling means being configured to aseptically collect the raw material which is contained in a first container of a predetermined volume, and
A receiving chamber which has a confinable space and which is configured to allow decontamination thereof via the passage of at least one fluid in said confinable space, said receiving chamber being provided with:
a closable opening configured for the passage of said first container in said confinable space and, when closed, is configured to confine the receiving chamber,
a receiving means orientable in the confinable space and being configured to receive said first container and position it in a direction of said sampling means, the latter being configured to aseptically collect said solution contained in said first container and arranged to aseptically transfer it to a second container having a volume greater than the volume of said first container.

2. The aseptic sampling device according to claim 1, wherein said sampling means is adjacent to said confinable space of said receiving chamber before sampling, and is preferably in a confined and sealed environment.

3. The aseptic sampling device according to claim 1, wherein said sampling means is separated from said receiving chamber via a resealable element selected from a group comprising a septum, a membrane, and a solenoid valve.

4. The aseptic sampling device according to claim 1, wherein, during said sampling, a part of said sampling means is located in the confinable space of the receiving chamber.

5. The aseptic sampling device according to claim 1, wherein said sampling means is located in a sampling chamber which has a guide means of said sampling means.

6. The aseptic sampling device according to claim 5, wherein said guide means is arranged to move said sampling means along a median axis of said sampling chamber towards said receiving chamber.

7. The aseptic sampling device according to claim 5, wherein said guide means has a groove which extends longitudinally on an outer surface of said guide means to slide said sampling means so that one of the ends of said sampling means is introduced into said receiving chamber.

8. The aseptic sampling device according to claim 1, wherein said sampling means has a body arranged to allow the passage of said raw material and which is provided at one of its ends with a suction means, preferably a needle or a line.

9. The aseptic sampling device according to claim 5, wherein said sampling chamber is confined and sealed.

10. The aseptic sampling device according to claim 1, wherein said receiving chamber has at least a part of its surface made of flexible material, said part being optionally adjacent to said receiving means, which is arranged to pass through said at least a part of the surface of the receiving chamber made of flexible material.

11. The aseptic sampling device according to claim 1, comprising a second container adjacent to said receiving chamber and which is connected by said sampling means to said receiving chamber.

12. The aseptic sampling device according to claim 11, wherein said second container has a volume greater than that of the first container and is arranged to allow culturing, when said raw material collected from said first container is transferred into said second container.

13. The aseptic sampling device according to claim 1, wherein said receiving means has a first part which extends into said confinable space and a second part which extends outside said receiving chamber in a form of a guide means, preferably along a median axis which passes through said receiving chamber.

14. The aseptic sampling device according to claim 1, wherein said part in a form of a guide means is in fluid communication with said receiving chamber.

15. The aseptic sampling device according to claim 1, wherein said receiving chamber is configured to be in fluid communication with at least one of the following elements or taken in combination:
A means for supplying at least one decontamination fluid,
A means for supplying at least one rinsing fluid,
A means for supplying at least one drying fluid,
A first means for removing at least one fluid, said removal means being optionally directly connected to one of the ends of said receiving means,
And a second means for removing drying fluid.
